(19) United States
(12) Patent Application Publication
(10) Pub. No.: US 2016/0000646 A1
(43) Pub. Date: Jan. 7, 2016

(54) Application module for a handheld device for the repeated application of an application element to a human skin or to an animal skin, and handheld device

(71) Applicant: MT.DERM GmbH, Berlin (DE)

(72) Inventor: Dirk Scherkowski, Berlin (DE)

(73) Assignee: MT.DERM GMBH, Berlin (DE)

(21) Appl. No.: 14/792,897

(22) Filed: Jul. 7, 2015

(30) Foreign Application Priority Data
Jul. 7, 2014 (EP) 14 176 018.1

(51) Int. Cl. A61H 23/02; A61M 37/00

(52) U.S. Cl. CPC A61H 23/0254; A61M 37/0076

(57) ABSTRACT

A handheld device for the repeated application of an application element to human or animal skin, comprising a housing; an application device arranged in the housing and an application element arranged on an application element receiving device. The application element receiving device with the application element is displaceable between an extended position and a retracted position. A coupling device functionally couples to pick up a drive force provided by a drive module An applicator, formed on the housing, rests in a skin region to be treated. The application element moves forwards and backwards within an application element chamber having an application element opening associated with the distal end of the application element. The application element opening opens the application element chamber towards the applicator, or is arranged in the region of the applicator. A suction chamber, separate from the application element chamber, provides a suction connection.

The present disclosure relates to an application module for a handheld device for the repeated application of an application element to a human skin or to an animal skin and to a handheld device.

### BACKGROUND

Such handheld devices are used to repeatedly apply an application element to a skin. For example, devices for locally puncturing a human skin or an animal skin are known. The skin can be locally treated, in particular punctured.

Handheld devices in which the application element is designed as a puncturing element can be used by operating personnel for applying a dye for tattooing and/or for applying permanent make-up in the region of the skin surface. Furthermore, the introduction of cosmetic or medical agents or substances by way of/into the skin is possible with such devices in that the skin is locally punctured. Moreover, such devices can be used without any substance being introduced, for example for the purpose of skin stimulation.

A handheld device for locally puncturing a skin is, for example, known from printed publication DE 299 19 199 U1. The known handheld device comprises a handle piece, a drive device and an application element that in operation by means of the drive device is moved forwards and backwards relative to a needle nozzle, wherein at least two disconnectably interconnected modules are provided, and in that one of the two modules is designed as a reusable base module with an integrated drive device. The other of the two modules is a sterilised one-way module in which in the known handheld device all the components that can be contaminated by the bodily fluids of a client are integrated. In this manner the handheld device is provided in the form of two modules, wherein one of them, namely the one-way module, can be exchanged after use, while the other module, which comprises the drive device, is reused. With the use of the one-way module the hygienic conditions during the application of a tattoo and/or of permanent make-up are improved, because all the components that can potentially be contaminated by the client's bodily fluid issued during treatment are replaced. In this way the need to replace the entire handheld device is obviated.

From the document EP 1 495 782 A1 a drive module for a device for locally puncturing a human skin or an animal skin is known in which drive module a drive device is provided by means of which a drive movement can be generated and, furthermore, a conversion mechanism, coupled to the drive device, is provided by means of which the rotary drive movement is converted to a forwards and backwards movement that can be coupled into an application device that locally punctures the skin so that a repeating movement of a puncturing means becomes possible.

From the document EP 0 006 395 A1 a tattooing device for marking animals is known, which tattooing device has a top unit that comprises at least one vacuum connection as well as suction openings on the outside surface, which top unit is arranged between the needle holders with tattooing needles. The needle holders are connected to control devices and to drives in the tattooing device. Marking the animals takes place in that a device is brought into contact with the skin surface of the animal. By way of the vacuum connection, the annular duct and holes, negative pressure is generated at the suction openings. The skin is aspirated to the outside surface of the grid or top unit, after which the placement of the marks takes place by means of the tattooing needles. After the mark has been applied, the negative pressure is released, and the device can be separated from the skin.

A handheld device for puncturing a human skin or an animal skin is known from document EP 2 682 146 A1. The handheld device comprises a housing, a needle module, a drive device which is arranged in the housing and which is suitable for repeatedly providing a feed movement, a puncturing device formed in the housing by means of a needle and coupled to the drive device either directly or by way of a coupling device in such a manner that the feed movement provided by the drive device can be introduced to the puncturing device, a housing opening relative to which in operation the needle moves forwards and backwards, and a puncturing-depth adjustment device that is coupled to the puncturing device in such a manner that the puncturing-depth adjustment device is displaceable relative to the tip of the needle and in operation during the forwards and backwards movement of the needle is moved together with said needle.

Document WO 2010/016635 A1 discloses a suction adapter for a syringe. The suction adapter is connected to a syringe needle with an air suction unit for generating a negative pressure on a skin. The suction adapter comprises a first dividing element and a second dividing element. A through-hole connects the first dividing element to the second dividing element in such a manner that a needle moves through the through-hole and projects from the top of the second dividing element.

Document EP 2 633 882 A1 relates to a module for skin surgery. The module comprises a main body whose two sides are open, and a subassembly that moves to and fro in the interior, which subassembly is installed in the main body. The subassembly can be moved forwards and backwards in the longitudinal direction of the main body by means of a screw-type coupling. In the main body a needle arrangement is formed that comprises several needles.

### SUMMARY

It is the object to state an application module for a handheld device for the repeated application of an application element to a human skin or to an animal skin, as well as a handheld device, which support improved application to the skin to be treated.

An application module for a handheld device for the repeated application of an application element to a human skin or to an animal skin according to the claim 1, and a handheld device according to the claim 15 are provided. Embodiments form part of dependent subordinate claims.

According to one aspect, an application module for a handheld device for the repeated application of an application element to a human skin or to an animal skin is created, with the aforesaid comprising a housing and an application device arranged in the housing and comprising an application element arranged or accommodated in an application-means receiving device, wherein the application-means receiving device with the application element is displaceable between an extended position and a retracted position. In operation, in this manner a distal end of the application element can be made to establish contact with the skin and can be moved away from the skin again, whether or not puncturing the skin, for example for massage purposes. The application module comprises a coupling device that is designed to functionally couple, either directly or by way of one or several interposed coupling elements, to a drive module of the handheld device so that a drive force provided by the drive module can be picked up and provided at least for a drive of the application device in the application module. An applicator, formed on the housing, is provided that in operation comes to rest in a skin region to be treated. If the application module is used on the handheld device, the applicator comes to rest in the skin region in which the skin is to be punctured repeatedly. In operation the application element moves forwards and backwards in an application element chamber, either once only or repeatedly. The application element chamber comprises an application element opening, associated with the distal end of the application element, which application element opening opens the application element chamber towards the applicator or is arranged in the region of the applicator. The application module further comprises a suction chamber that is separate from the application element chamber. On the suction chamber a suction connection is formed by way of which a pneumatic suction force can be introduced into the suction chamber. The suction chamber communicates with a suction opening that is arranged in the region of the applicator. In operation, the suction opening, which can comprise one or several breakouts (partial openings), is placed with the applicator onto the skin so that a skin section that is opposite the suction opening is aspirated, and optionally can also be drawn some way into the suction opening while the pneumatic suction force is applied by way of the suction connection to the suction chamber.

According to a further aspect, a handheld device for the repeated application of an application element to a human skin or to an animal skin with a drive module is created that is designed to provide a drive force. The handheld device further comprises an application module of the type mentioned above that functionally couples to the drive module, either detachably or non-detachably, so that the drive force provided by the drive module can be picked up and is provided in the application module at least for driving an application device.

The projection of the puncturing means relative to the application element opening can be adjustable.

As a supplement, in one embodiment it can be provided that an additional pneumatic suction force is made available by way of the application element chamber, wherein the pneumatic suction force can be introduced to the application element chamber, for example by way of a suction connection of said application element chamber. As an alternative or as a supplement, it can be provided that a wall section that divides the application element chamber from the suction chamber comprises one or several breakouts by way of which the pneumatic suction force applied to the suction chamber is also introduced into the application element chamber. In an alternative embodiment, in operation the application element chamber is not subjected to any suction force. Aspiration of the skin when the applicator is in place then takes place exclusively by means of the suction chamber, which in turn in this embodiment or in other embodiments can be designed as a single-chamber system or a multi-chamber system. Irrespective of the design as a single-chamber system or a multi-chamber system the suction opening can comprise one suction breakout or several suction breakouts.

The application element chamber can be of any shape and can comprise one or several sub-chambers. In operation, the application element can at least at times be arranged therein either entirely or only partly.

Any transfer of the suction force from the suction chamber to the application element chamber can be prevented, in particular, when the applicator has been placed onto the skin.

The application element can, at least in operation, in some sections also extend through the suction chamber, wherein a wall passage, of the application element, into the suction chamber can be designed so as to be sealed.

By means of the pneumatic suction force, which in operation is coupled to the suction chamber, when the applicator is in place the skin is firmly held for the puncturing means to puncture the skin. In this process it can be provided for the coupled-in suction pressure to be controlled depending on the position of the puncturing means (retracted/extended). For example, the suction force can be reduced or completely switched off when the application element is in its retracted position and/or moves from the extended position to the retracted position. Furthermore, easing or switching-off an initially persistent suction force after a predetermined number of puncturing movements can be provided.

The application module can be designed, in reoccurring repeated application procedures, for example puncturing procedures, to be operated at a repetition frequency of between approximately 5 and approximately 500 Hz (strokes per second), preferably between approximately 50 and approximately 250 Hz. The drive module is then designed to provide a corresponding drive movement.

The enlargement of the skin surface, which enlargement occurs during the possible drawing of the skin into the applicator, can cause preliminary tension of the skin and thus a reduction in evasion of the skin, which evasion is referred to as the “umbrella effect”, for example in needle treatments, in particular in the region of the front opening of the application-element means chamber. A further increase in the skin pre-tension during persistent suction pressure can be achieved by lifting the applicator surface from the original skin level, in particular, because due to easy lateral sliding of the applicator or of the applicator surface on the skin surface and vice versa automatic equalisation of the skin pre-tension in the entire lifted and drawn-in skin region can result, and consequently tympanum-like stretching of the skin in front of the opening of the application element chamber can be achieved, which ultimately leads to a more even treatment result, thus obviating the need for any tensioning with additional finger force, which is a known cause of undesirable haematoma formation.

If the application element is designed as a puncturing element, it can comprise one or several individual needles, one or several needle groups and/or one or several needle plates. The latter as such has been disclosed, for example, in document EP 2 462 979 A1. In the context of these embodiments, the application element can overall also be referred to as a “puncturing needle” that is arranged on a needle receiving device.

In one embodiment the radius of the needle or of the needles can be selected in such a manner that during skin treatment the emphasis is on skin stimulation by means of a mechanical load.

It can be provided that the application element opening and the suction opening are arranged adjacent to each other in the region of an application surface formed on a distal end of the applicator. The application surface can extend between front faces of the applicator. In one embodiment the suction opening is arranged in the application surface, whereas the application element opening can be offset back relative to this.

In one embodiment it can be provided that the suction chamber is at least in part surrounded by a flared wall section that at least in part on the rear extends away from the suction opening. The flared embodiment of the wall can extend along an outside wall of the applicator so as to be circumferential, continuous or interrupted. If the suction opening comprises several suction breakouts, they can entirely or partly be surrounded by the flared wall.

It can be provided that the cross section of the suction chamber tapers off in a chamber section opposite the suction opening. Starting from the suction opening the tapered section can extend all the way to the suction connection or part of this distance. Wall sections that face the suction chamber, for example in the region of the tapered section, can be designed to be planar and/or curved, for example curved towards the interior of the suction chamber.

The suction opening can at least in part be surrounded by one or several wall sections made from a flexible material. The flexible material can be elastically resilient. For example a rubber material, a silicon material, or some other plastic material can be used.

The application element chamber can comprise a lateral opening. The lateral opening can comprise one or several breakouts, in a round shape and/or in a slit shape. An opening can extend starting from the application surface.

The suction chamber can surround the application element chamber on several sides. In this embodiment or in other embodiments it can be provided that the suction opening entirely or partly surrounds the application element opening, or vice versa. In one embodiment the application element opening can be elongated, wherein, on the two opposite sides of the application element opening, suction breakouts of the suction opening extend. Moreover, in one embodiment a suction chamber can be arranged on the inside, wherein on the outside one or several suction openings are formed.

An improvement can provide that the coupling device is designed to provide a mechanical, a magnetic, an electrical and/or a pneumatic drive force at least for a drive of the application device in the application module. By way of the coupling device one or several drive forces for the functional components can be coupled into the application module. For example, a mechanical drive force can be transmitted by way of a coupling rod assembly. As an alternative or as a supplement it can be provided that a pneumatic drive force is coupled by way of a fluid line, either a positive pressure and/or a negative pressure, for example a pneumatic drive force. As a supplement or as an alternative an electromagnetic coupling can be provided, for example by means of one or several magnets that are associated with each other and that can comprise electrical magnets and/or permanent magnets. If, for example, a linear drive movement and a rotary drive movement are transmitted from the drive module to the application module, then in the application module a component for movement decoupling can be provided in such a manner that, for example, the rotary drive movement is used for driving a vacuum pump in the application module, and the linear drive movement is used for displacing the application-means receiving device. However, it is also possible for the linear drive movement to be used for generating the pneumatic suction force, for example in that by means of it a vacuum pump is driven.

The coupling device can be designed to pick up a rotary and/or a straight-line drive movement. For example, the mechanical drive movement can be transmitted, by way of a connecting rod element or a rod assembly, from the drive module to the application module. In this embodiment or in other embodiments in the drive module a drive force can be provided, for example my means of an electric motor. Either in the drive module or in the application module the rotation of a drive shaft can be converted to the application module, for which purpose a conversion mechanism is used, as is known as such in various embodiments.

One embodiment provides that the coupling device is designed to pick up in a non-contacting manner the drive force provided by the drive module. Non-contacting pickup can, for example, be implemented with the use of a magnetic coupling with facing magnets outside and inside the housing of the application module. In this embodiment or in other embodiments the housing can be formed, at least in the region of the pickup of the drive force, as a closed housing. In this embodiment or in other embodiments transmission of the drive movement (drive force) in the application module by means of a magnetic coupling can take place as a linear movement or as a rotary movement. To this effect one or several magnets can be arranged in the application module so that they are rotatable or linearly displaceable in order to follow a corresponding movement of associated magnet elements outside the housing of the application module.

The coupling device can be connected to a conversion mechanism that is arranged in the housing and that is designed to convert one type of drive movement to some other type of drive movement, which is coupled to the application device, which drive movement differs from the type of drive movement. For example, by means of the conversion mechanism a rotary movement can be converted to a straight-line movement or to a linear movement. Furthermore, a reverse conversion of the type of drive movement can be provided. Conversion mechanisms as such, in particular in an embodiment for handheld devices for repeatedly puncturing a skin, are known in various embodiments. For example, a combination of wobble plate and plunger is known by means of which a rotary drive movement can be converted to a linear movement for driving the application device.

In the housing a pumping unit can be arranged that is in fluidic communication with the suction connection of the suction chamber in such a manner that a suction pressure generated by the pumping unit can be applied to the suction chamber. The pumping unit in the housing can be designed as a vacuum pump. The pumping unit can be a piston pump, wherein an air-conveying piston movement can be carried out based on a mechanical, a magnetic, or a pneumatic drive force. As an alternative or as a supplement, for generating a suction force by means of the pumping unit in the housing, the introduction of a suction force, for example negative pressure, from outside the application module from an external source can be provided. To this effect the housing can comprise a pneumatic connection by way of which a positive pressure and/or a negative pressure can be introduced. The pneumatic connection can be in fluidic communication with the suction connection of the suction chamber, either directly or by way of connecting elements, for example one or several valves.

An improvement can provide that in the housing a fluid reservoir is formed that is designed to receive a substance to be introduced. The fluid reservoir can, for example, embrace the interior of the housing to the extent that said interior has not been taken up by other functional components. It can be provided that in this design the received fluid (substance) surrounds other functional components arranged in the interior. Furthermore, a fluid reservoir that is delimited vis-à-vis the interior can be provided. By way of fluidic communication the received fluid moves from the reservoir to the application element on whose surface it can be output as a result of the forwards and backwards movement. It can be provided for the fluid reservoir to be subjected to pressure that supports the output of the fluid in operation. The fluid can, for example, be a dye for producing a tattoo or for producing permanent make-up. However, medical and/or cosmetic substances, too, can be provided for introduction into the skin and/or for application to the skin surface. If the application element is formed with a hollow needle, as a supplement or as an alternative the substance can be output through the hollow space of the hollow needle, be it exclusively or supplementary through the aforesaid. It can be provided, by means of the suction effect provided by way of the suction chamber, to receive back in (suck into) the application module any remaining fluid that has been output and that has not been taken up by the skin, and to convey said fluid to the fluid reservoir. In this process a stable foam can form in the case of a foamable fluid, which foam wets the application element and/or the skin, thus checking or preventing unintended issuing of the fluid. With a correspondingly adapted foaming behaviour of the fluid it is possible in this manner to also control the dosing strength and the consumption of the active substance.

The application device can be a puncturing device that on the application element receiving device, which is designed as a puncturing-element receiving device, comprises the application element, which is designed as a puncturing element. On the distal end of the puncturing element, which can comprise one or several needles, a puncturing tip is arranged that is designed to puncture the skin. In this case, in the puncturing means chamber a needle opening or puncturing means opening is associated with the puncturing tip.

As an alternative, the application device can be a non-puncturing application device in which on the application element receiving device a non-puncturing application element is arranged that is designed to be applied to the skin in a non-puncturing manner, for example for massaging the skin. The distal end of the application element or treatment element then does not comprise a puncturing tip but instead is somewhat rounded, comprising, for example, a ball shape or spherical shape or a flat front face.

It can be provided that the application element receiving device is designed to allow tilting and/or rotating of the application element, in particular if the application element is designed as a non-puncturing application element. In operation, tilting/rotating of the application element can then, for example, occur when said application element is pressed against the skin. In the case of a non-puncturing application element, as an alternative or as a supplement to forwards and backwards movement, it can be provided for said application element, in operation, to be rotated on an axis that extends in the longitudinal direction of the housing of the application module.

The applicator can be detachably arranged on the housing. An assembly kit comprising an application module and several different applicators can be provided. Such a product makes it possible for the user to set up the application module for various applications by affixing a respectively desired applicator. The applicators can, for example, differ in terms or their size and/or their shape, in particular in terms of the size and/or shape of an application surface.

By means of a handheld device, which comprises a drive module and an application module that can be coupled to the aforesaid, a method for the repeated application of an application element to a human skin or to an animal skin is implementable. In operation, the application-means receiving device with the application element is displaced between an extended position and a retracted position in such a manner that a distal end of the application element is brought into contact with the skin and is moved away from the skin, either puncturing the skin or not puncturing the skin, for example for massage purposes. An applicator formed on the housing of the application module is provided, which applicator in operation comes to rest in a skin region to be treated. With the use of the application module on the handheld device the applicator comes to rest in the skin region in which the skin is to be treated repeatedly, either in a puncturing or non-puncturing manner In operation the application element moves forwards and backwards, once only or repeatedly, in an application element chamber. The application element chamber comprises an application element opening, associated with the distal end of the application element, which application element opening opens the application element chamber towards the applicator or is arranged in the region of the applicator. The application module further comprises a suction chamber that is separate from the application element chamber. On the suction chamber a suction connection is formed, by way of which a pneumatic suction force is introduced into the suction chamber. The suction chamber communicates with a suction opening that is arranged in the region of the applicator. In operation the suction opening with the applicator is placed onto the skin so that a skin section situated opposite the suction opening is aspirated, and optionally some of the skin can be drawn somewhat into the suction opening when the pneumatic suction force has been applied to the suction chamber by way of the suction connection. Parallel in time to the skin being aspirated, the application element is extended.

### DESCRIPTION OF EMBODIMENTS

Below, further exemplary embodiments are explained in more detail with reference to the figures of a drawing. The following are shown:

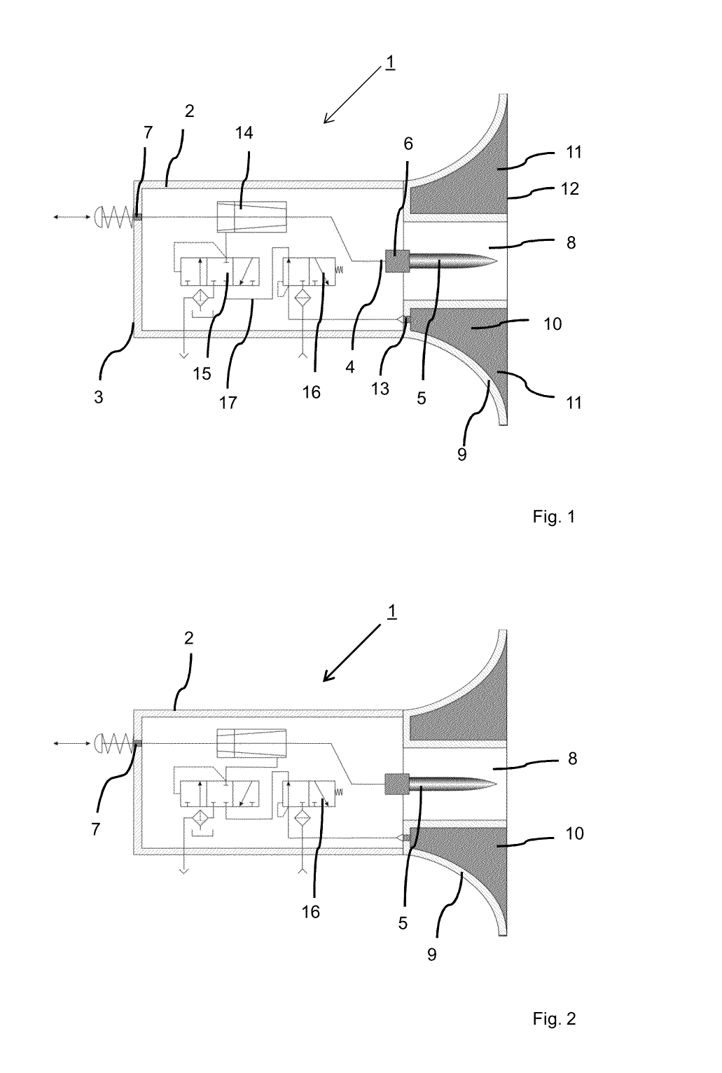

FIG. 1 a diagrammatic cross-sectional view of an application module for a handheld device for the repeated puncturing of a human skin or of an animal skin, in which a linear drive movement is coupled in,

FIG. 2 a diagrammatic cross-sectional view of a further application module in which a linear drive movement is coupled in,

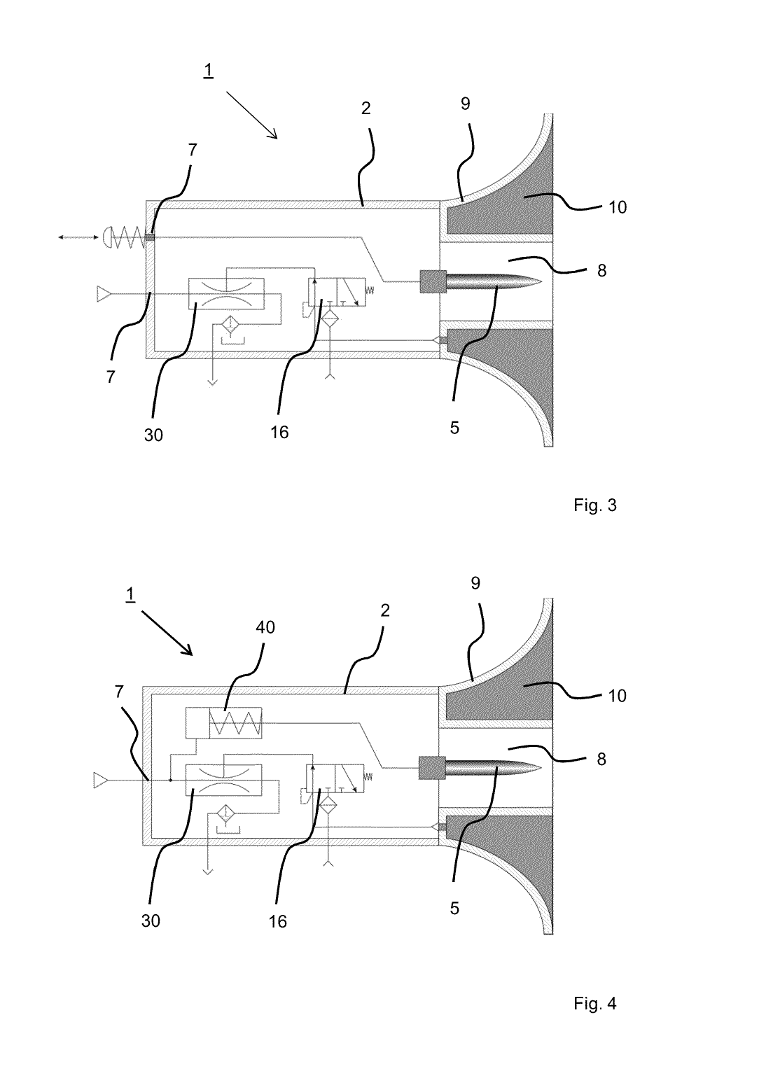

FIG. 3 a diagrammatic cross-sectional view of an application module in which apart from a linear drive movement a positive pressure from the outside is provided on the application module,

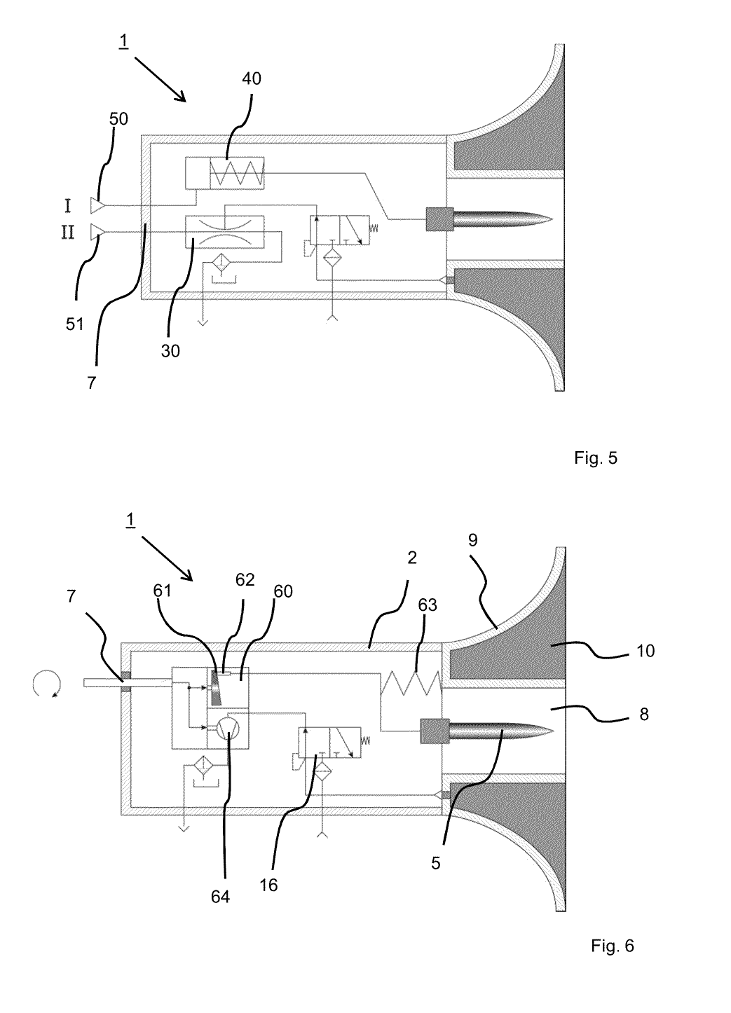

FIG. 4 a diagrammatic cross-sectional view of another application module in which apart from a linear drive movement a negative pressure from the outside is provided on the application module,

FIG. 5 a diagrammatic cross-sectional view of an application module in which several pneumatic connections are provided,

FIG. 6 a diagrammatic cross-sectional view of an application module in which a rotary drive movement is coupled in,

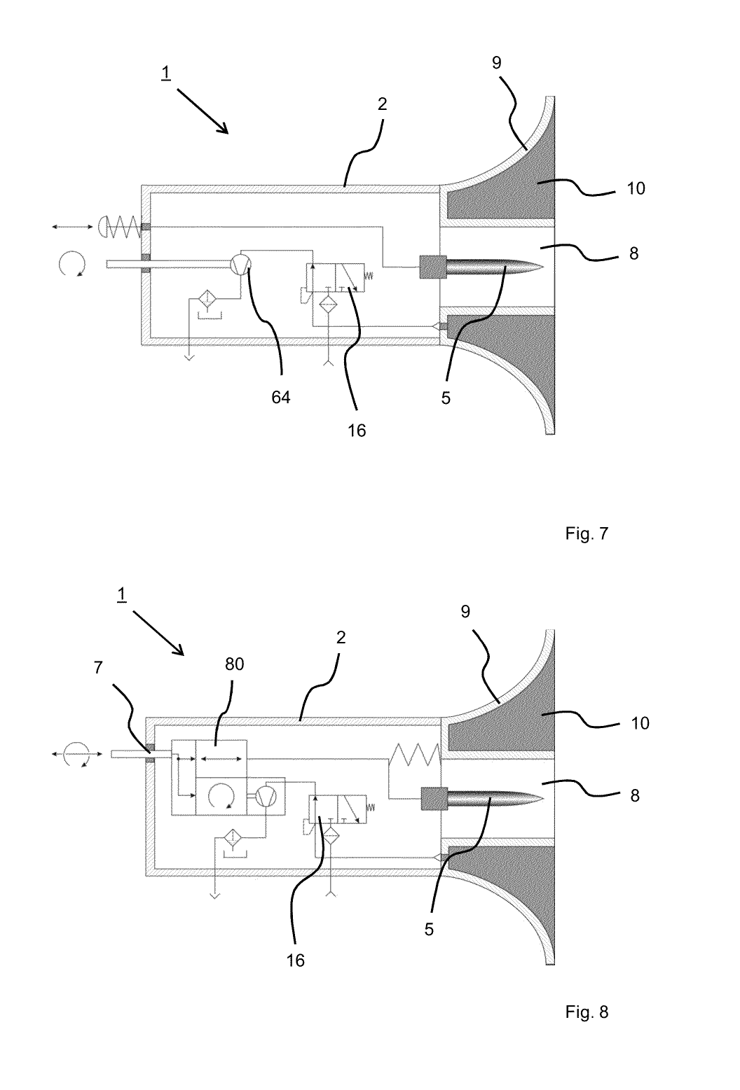

FIG. 7 a diagrammatic cross-sectional view of an application module in which a linear drive movement and a rotary drive movement are coupled in,

FIG. 8 a diagrammatic cross-sectional view of an application module in which by way of one and the same coupling both a linear and a rotary drive movement are coupled in and are decoupled in the application module,

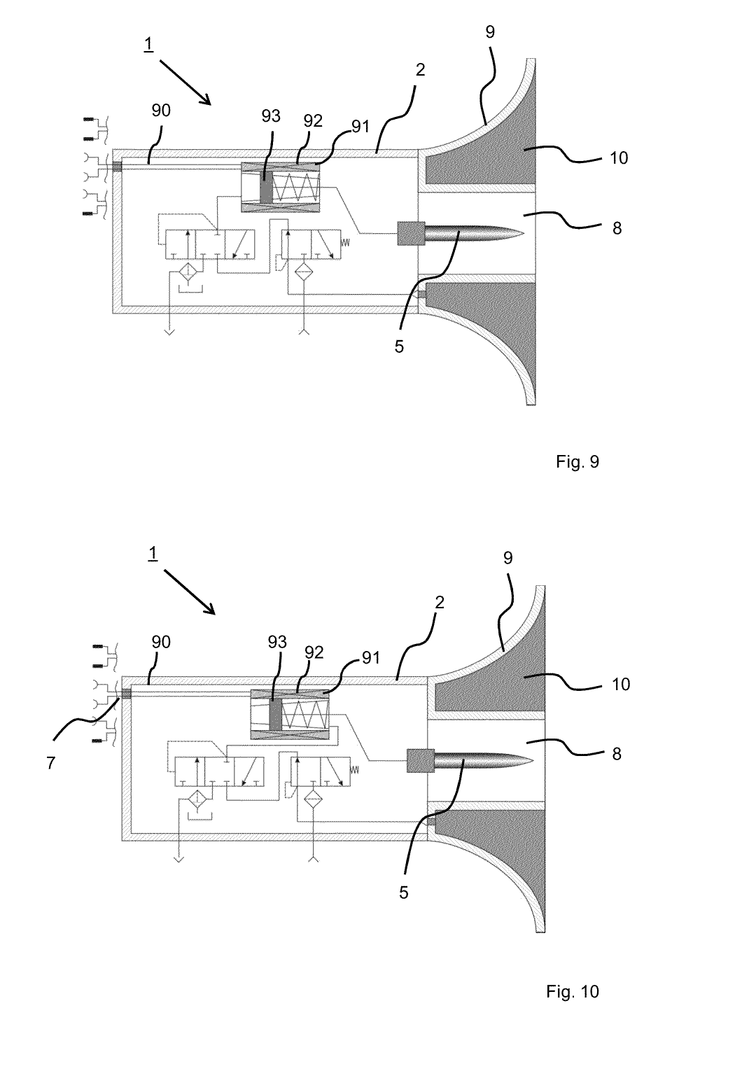

FIG. 9 a diagrammatic cross-sectional view of an application module in which electrical drive energy is coupled in,

FIG. 10 a diagrammatic cross-sectional view of another application module in which electrical drive energy is coupled in,

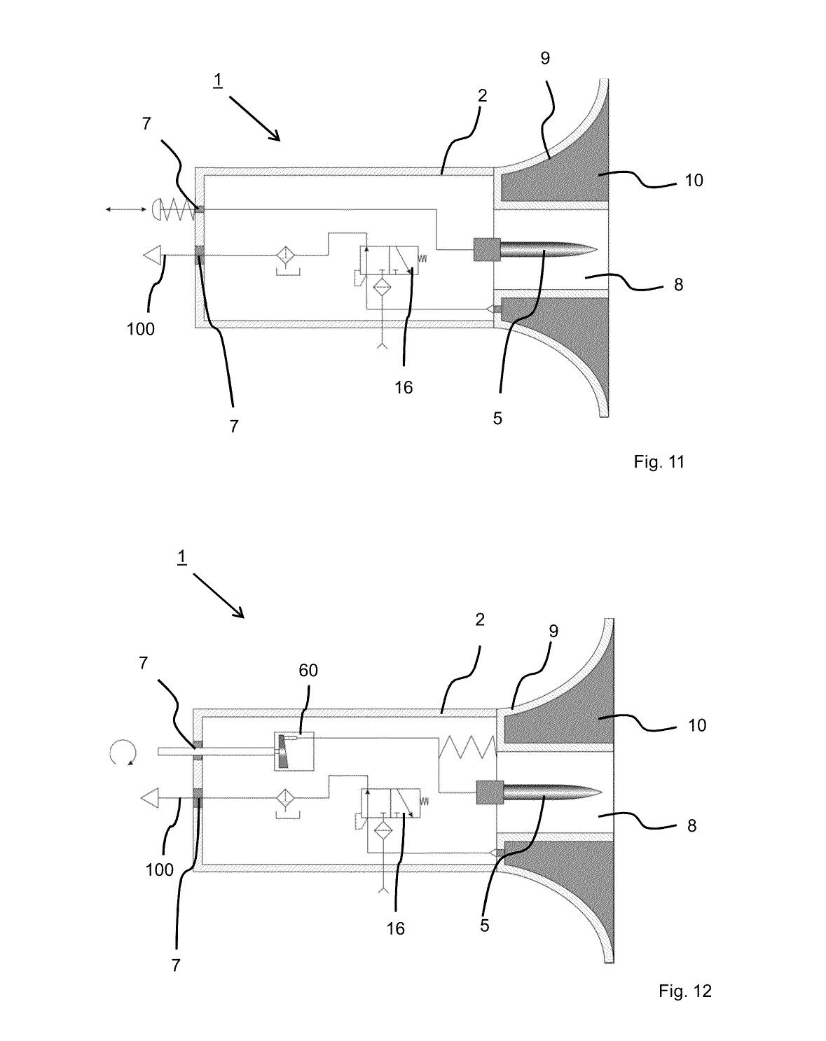

FIG. 11 a diagrammatic cross-sectional view of an application module in which a linear drive movement is coupled in, and by way of a pneumatic connection from the outside a negative pressure is coupled in,

FIG. 12 a diagrammatic cross-sectional view of an application module in which a rotary drive movement is coupled in, and by way of a pneumatic connection from the outside a negative pressure is coupled in,

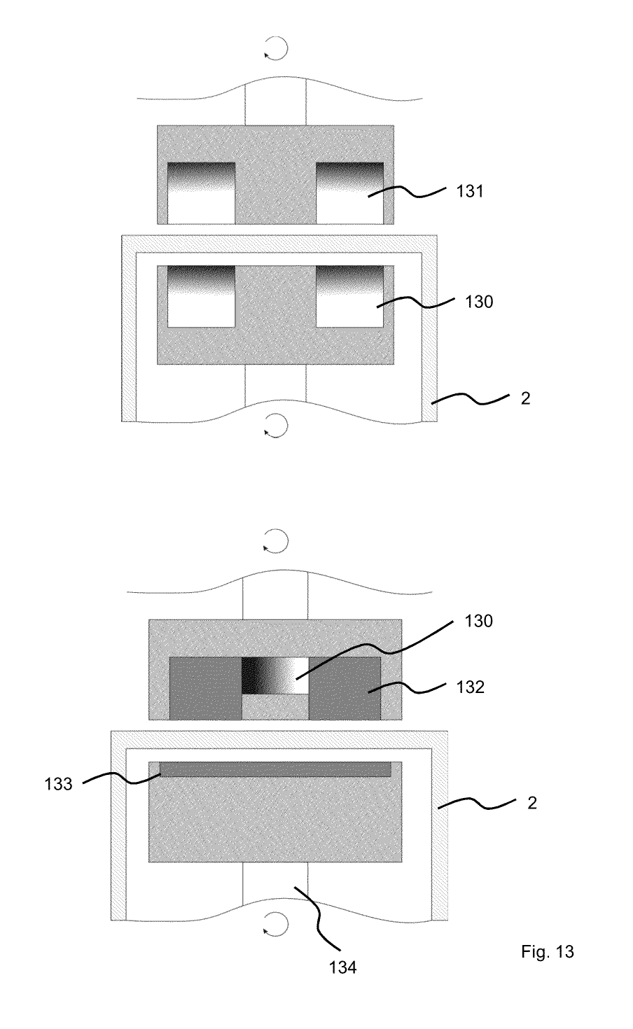

FIG. 13 a diagrammatic view of a coupling device for the non-contacting pickup of a rotary drive movement by means of a magnetic coupling,

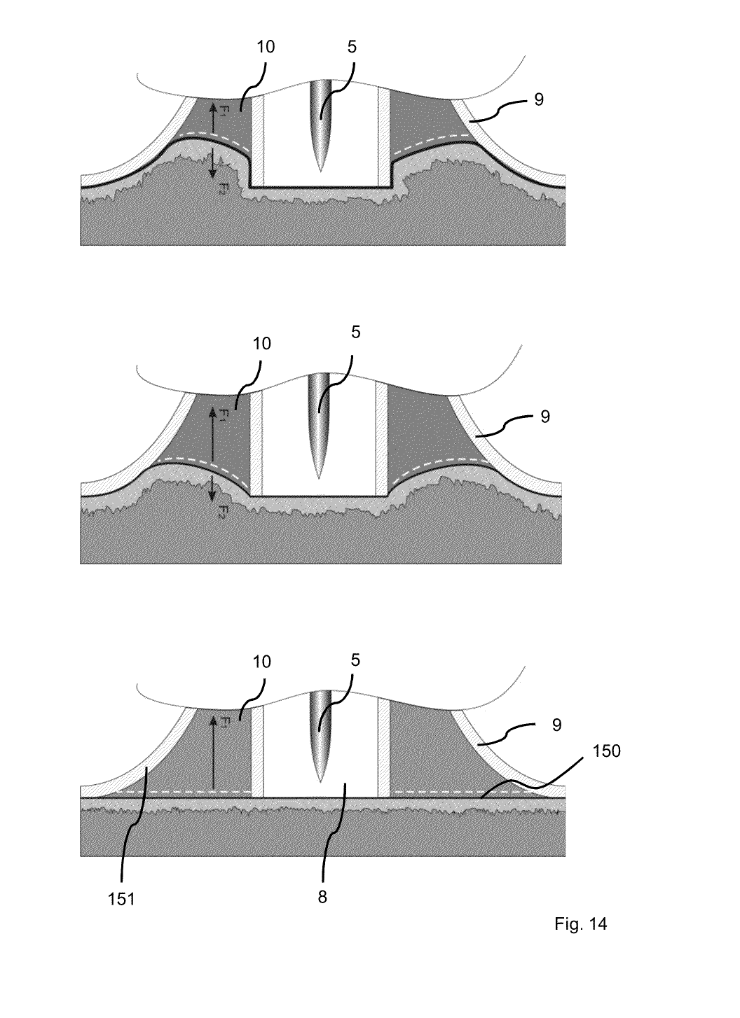

FIG. 14 a diagrammatic cross-sectional view of a front section of an applicator for explaining aspiration of an upper layer of the skin,

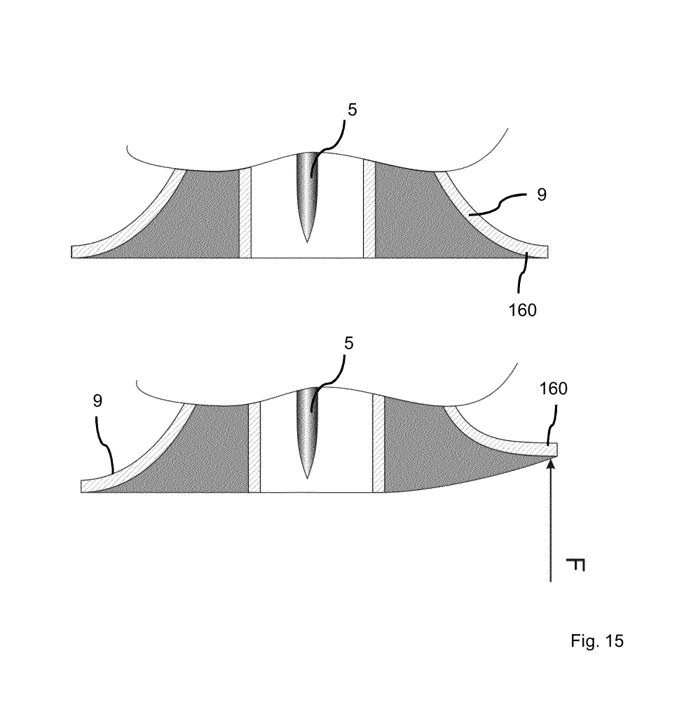

FIG. 15 a diagrammatic cross-sectional view of a front section of an applicator, wherein a wall section comprises a flexible material,

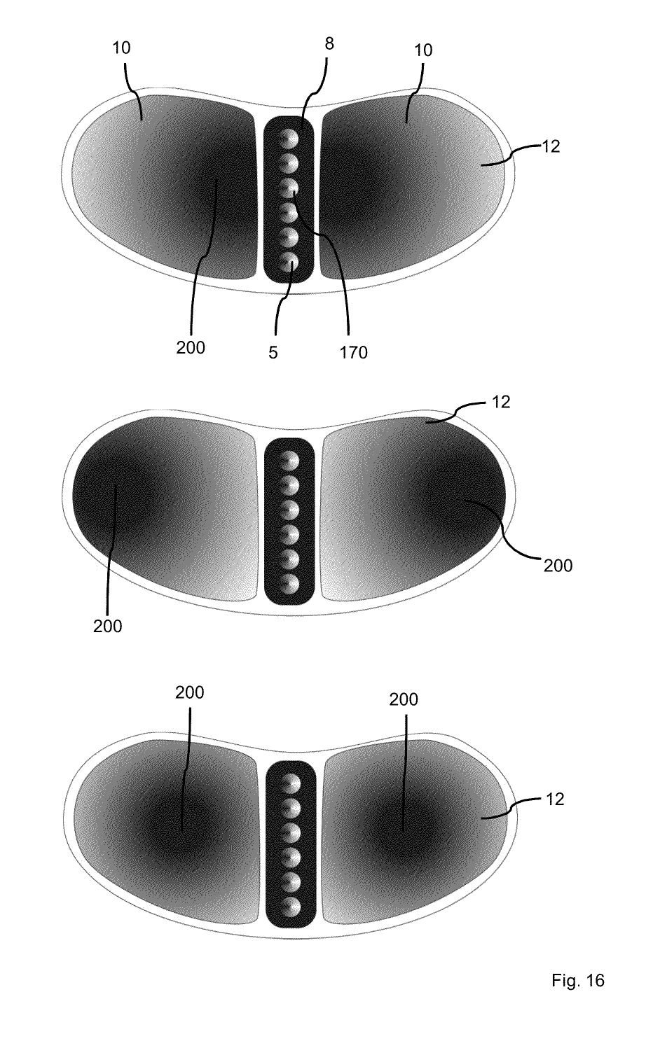

FIG. 16 a diagrammatic top view of an application surface of an applicator, wherein the application surface is kidney-shaped,

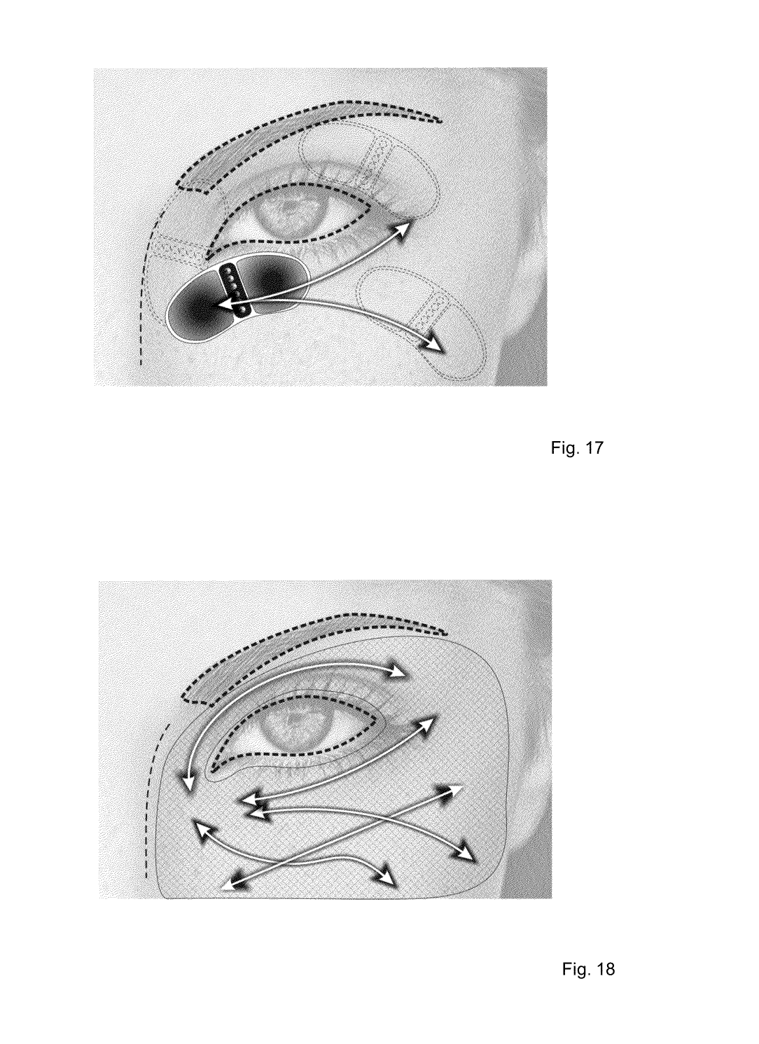

FIG. 17 a diagrammatic view for the placement of an applicator in the region of a zone around the eyes,

FIG. 18 a diagrammatic view for the placement of an applicator in the region of a zone around the eyes, wherein an area to be treated is cross-hatched,

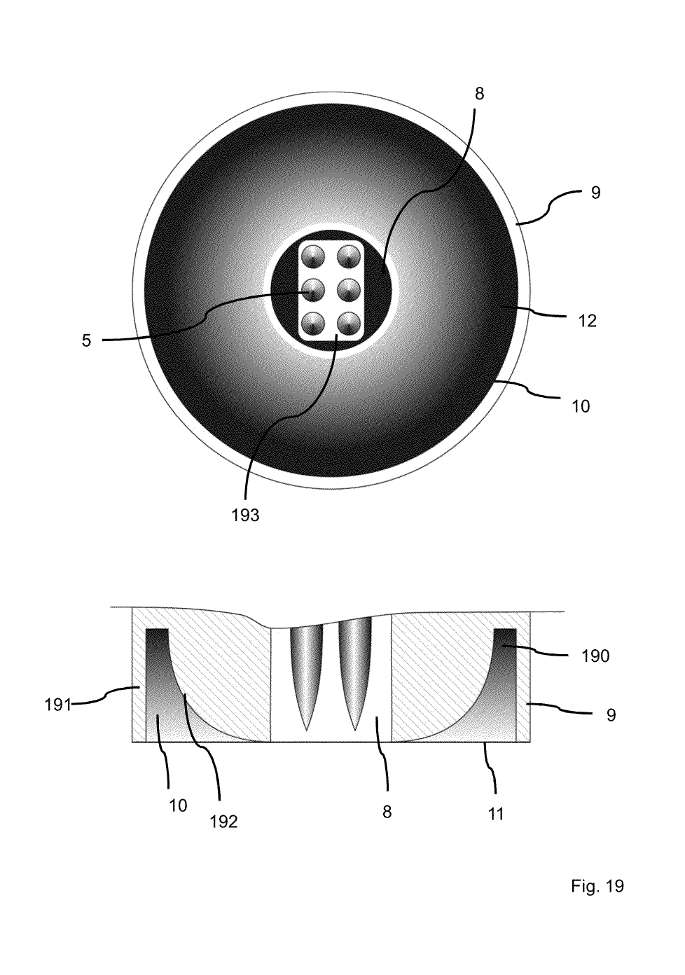

FIG. 19 a diagrammatic cross-sectional top view of an application surface on an applicator,

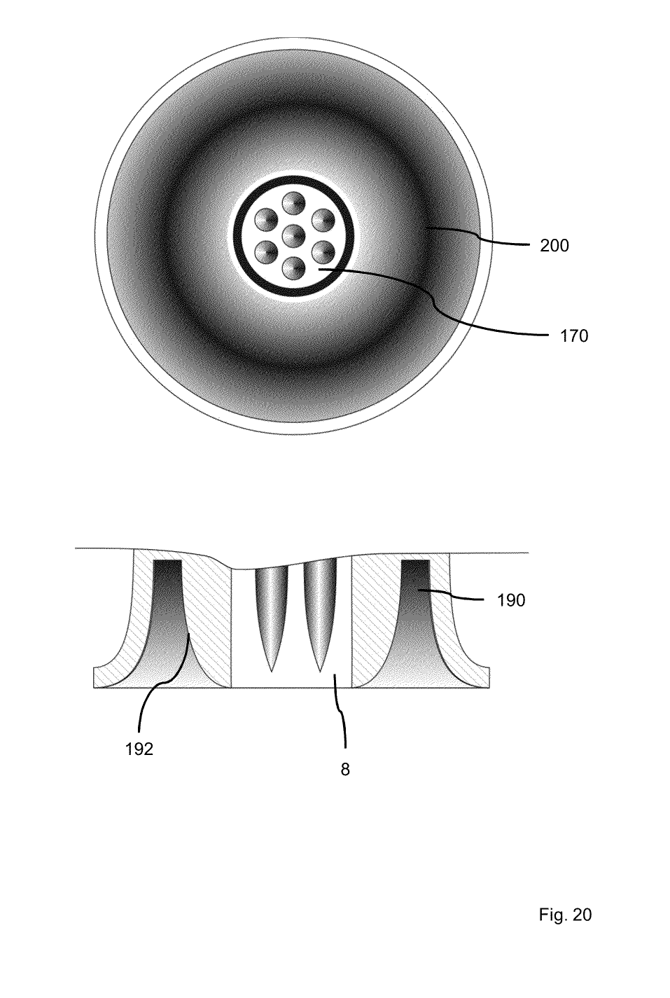

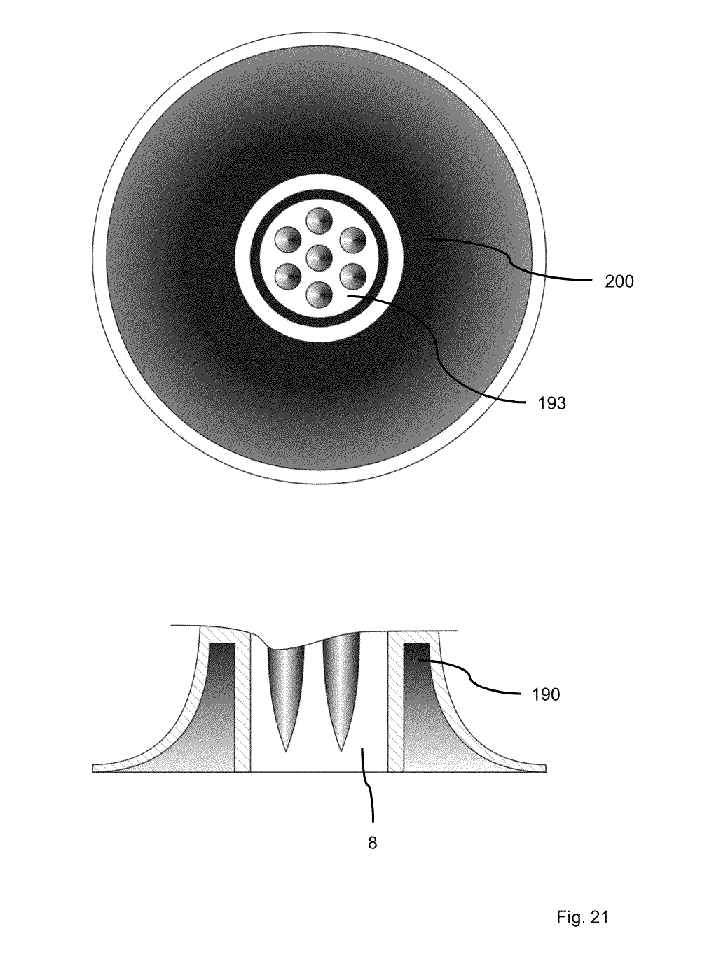

FIG. 20 a diagrammatic cross-sectional top view of another application surface on an applicator,

FIG. 21 a diagrammatic cross-sectional top view of a further application surface on an applicator,

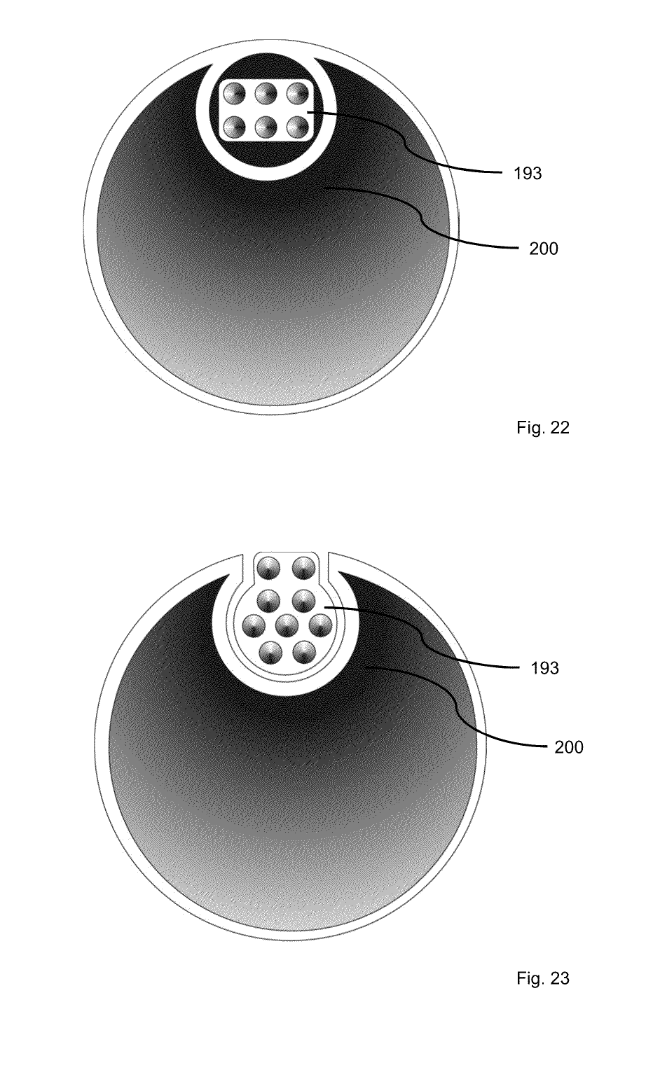

FIG. 22 a diagrammatic view of an application surface on an applicator with a laterally arranged puncturing means,

FIG. 23 a diagrammatic view of another application surface on an applicator also with a laterally arranged application element, and

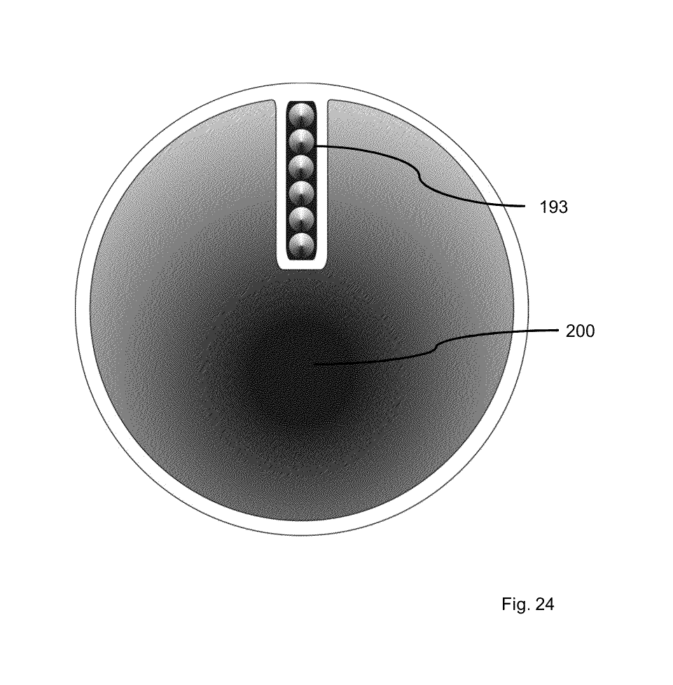

FIG. 24 a diagrammatic view of a further application surface on an applicator with a laterally arranged puncturing means.

FIG. 1 shows a diagrammatic view of an application module 1, which in the exemplary embodiment shown is designed as a puncturing module, for a handheld device for the repeated puncturing of a human skin or an animal skin. A housing 2 is provided in which a rear surface 3 can face a drive module (not shown) of the handheld device when the application module 1 functionally couples, either detachably or non-detachably, to the drive module of the handheld device. A detachable attachment can, for example, be established by means of a screw connection, a clamping connection and/or a plug-in connection. The application module 1 can be designed as a one-way module. In particular, the housing 2 can comprise a plastic material, and can, for example, be designed as an injection-moulded part.

In the housing 2 an application device 4 with an application element 5 is arranged that is received on an application-means receiving device 6. In operation, in other words when the application module 1 is arranged on the drive module of the handheld device for puncturing the skin, by way of a coupling device 7 a drive force provided by the drive module, in particular as a drive movement, is coupled into the application module 1 in order to carry out a forwards and backwards movement of the application-means receiving device 6 with the application element 5 arranged thereon so that the application-means receiving device 6 with the application element 5 is displaced between a retracted position and an extended position in the longitudinal direction of the housing 2. In the embodiment shown in FIG. 1, which also applies to the embodiment in FIG. 2, by way of the coupling device 7 a linear movement is coupled-in which is transmitted to the application device 4.

The application element 5 is arranged in an application element chamber 8 which in the embodiment shown is formed in an applicator 9. The application element chamber 8 is separate of a suction chamber 10 which according to FIGS. 1 and 2 in the embodiments shown extends on both sides of the application element chamber 8. The suction chamber 10 opens forwards by way of a suction opening 11 which in turn is situated in an application surface 12 at the front of the applicator 9.

In operation of the handheld device, in the region of the application surface 12, the applicator 9 is placed onto the skin to be treated. By way of a suction connection 13, a suction pressure guided from a vacuum pump 14 and by way of a valve device 15 as well as a further valve device 16 is applied to the suction chamber 10 so that in that location the skin surface is aspirated. Depending on the position of the valve device 15 and of the further valve device 16 the negative pressure is or is not applied to the suction connection 13. This takes place when fluidic communication 17 is connected through from the vacuum pump 14 to the suction connection 13.

In the design of the application module 1 in FIG. 2 conveyance of air takes place during the retraction movement of the application device 4, whereas in the embodiment in FIG. 1 this takes place during the extension movement. The drive movement for the application-means receiving device 6 and the movement for conveying air in the vacuum pump 14 are coupled movements. For example, one or several rod elements, which in turn are connected to the application-means receiving device 6, couple to a piston of the vacuum pump 14 so that the piston movement can be coupled into the application-means receiving device 6 in the longitudinal direction of the housing 2.

In FIGS. 3 to 12 the same reference characters as in FIGS. 1 and 2 are used for identical features.

In the design of the embodiment of the application module in FIG. 3, in a manner comparable to that of the design in FIGS. 1 and 2, a linear drive movement is coupled into the application module 1 by way of the coupling device 7. In order to generate the negative pressure for the suction chamber 10 an injector 30 is supplied with positive pressure by an external source (not shown), which injector 30, when subjected to the flow of pneumatic gas, by means of the known Venturi effect provides a negative pressure. The embodiment in FIG. 4 is of the same design in this respect, wherein in it the pneumatic pressure provided externally is used to drive a component 40 with a pneumatic cylinder so that in this manner the drive movement necessary for the application device 4 is provided.

The embodiment of the application module 1 according to FIG. 5 operates in a similar manner, wherein the injector 30 and the component 40 with the pneumatic cylinder are now supplied by way of separate connections 50, 51 which are, for example, separate compressed pressure connections.

In the embodiments of the application module 1 in FIGS. 6, 7 and 8 in each case a rotary drive movement is coupled into the application module 1. In the embodiment in FIG. 6 the rotary drive movement drives a conversion mechanism 60 by means of which the rotary drive movement is converted to a linear drive movement in the longitudinal direction of the housing 2 in order to drive the application device 4. To this effect in the exemplary embodiment shown the conversion mechanism 60 is formed with a wobble plate 61 and a plunger 62. A return spring 63 ensures the retraction movement of the application-means receiving device 6 with the application element 5. Furthermore, by means of the rotary drive movement a vacuum pump 64 is operated.

In FIG. 7 as a supplement to the rotary drive movement a linear drive movement is coupled in, comparable to the embodiments in Figs Ito 3. The conversion mechanism 60 is then omitted.

In the embodiment of the application module 1 in FIG. 8, rotary and linear drive movements are jointly coupled into the application module 1 in order to subsequently, by means of a decoupling device 80, decouple the two drive movements in the housing 2. For example, the rotary drive movement can be decoupled from the linear drive movement of a coupling part, arranged on the drive side, in the application module 1 in that on the one hand a pivotable part of a vacuum pump, for example of a vane cell compressor, with a shared rotary axis on the coupling part, which reaches through it and which is arranged on the drive side, which coupling part in its outermost prismatic contour is designed to transmit torque, couples in a stationary rotating manner while gliding relatively in the longitudinal direction in such a manner that during this process only the rotary movement is transmitted. On the other hand, decoupling of the linear drive movement can take place by means of a plunger, which couples to the face and in the rotary axis of the coupling part on the drive side, which plunger is linearly movable exclusively forward and backwards, with a preferably disappearing contact surface, which plunger is connected to the application device 4.

FIGS. 9 and 10 show diagrammatic views of embodiments of the application module 1 in which an electrical drive energy is provided by way of a connection line 90 in order to in this manner drive a pumping unit 91. In the aforesaid in the magnetic field of a coil 92, through which at least at times an electrical current flows, a pneumatic piston with a permanent magnet and/or a soft-iron core 93 is arranged that by means of electrical control is moved forwards and backwards in the longitudinal direction of the housing 2, as a result of which on the one hand the linear drive movement for the application device 4 and on the other hand the vacuum generation are carried out. While in the embodiment in FIG. 9 during the extension movement of the application device 4 air is conveyed, in the embodiment according to FIG. 10 this takes place during the return movement.

As an alternative it can be provided that the coil 92, instead of being arranged in the application module 1, is arranged outside the housing 2, optionally separately from the application module 1, for example integrated in the drive module (not shown), and in this manner a corresponding magnetic field that changes over time can act on the application module 1 in a non-contacting manner, which magnetic field can move a pneumatic piston as described above. For example, the housing 2 can be at least partly encompassed by a section of the drive module on which the coil is arranged so that the aforesaid extends along the housing 2, in particular laterally of the pneumatic piston with the permanent magnet and/or the soft-iron core 93. The coil body or a housing enveloping the coil 92 can rest on the external surface of the housing 2.

The embodiments in FIGS. 11 and 12 of the application module 1 show arrangements in which a linear drive movement (FIG. 11) or a rotary drive movement (FIG. 12) is coupled in, as has already been explained above. An external negative pressure is applied by way of a pneumatic connection 100 and, depending on the operation, is conveyed to the suction connection 13 by way of the further valve device 16.

FIG. 13 shows diagrammatic views of a coupling device for the non-contacting picking up of a rotary drive movement by means of a respective magnetic coupling in which inside and outside the housing 2 mutually associated magnets 130, 131 are arranged. In the region between the mutually associated magnets 130, 131 and adjacent to it the housing 2 is formed as a closed housing. In FIG. 13 in the bottom illustration a magnetic coupling is shown which on the drive side is formed with a permanent magnet 130 and a first part 132 of a magnetic inference system, wherein a completing part 133 of this system is situated on the rotary coupling part 134 in the application module 1.

In a manner analogous to the embodiments mentioned above, in which a rotary drive moment is coupled in a non-contacting manner, it can be provided to couple-in a linear drive movement in a non-contacting manner by way of a magnetic coupling. In this design one or several magnets (not shown) can move outside the housing 2 in its longitudinal direction. In the housing 2 the movement is picked up by means of associated magnets.

FIG. 14 shows a diagrammatic view of a front section of the applicator 9 when the latter has been placed onto a skin surface 150. The application element 5 is shown in a retracted position. By means of the application of the suction pressure by way of the suction connection 13 the skin surface is drawn into the suction chamber 10 (compare the middle illustration in FIG. 14) until a force equilibrium (compare top illustration in FIG. 14) has been achieved. In this design the skin surface 150 conforms to the surface 151 that is curved inwards towards the suction chamber 10.

FIG. 15 shows a view comparable to that of FIG. 14, wherein a wall 160 of the applicator 9 comprises an elastically resilient material so that during placement onto the skin surface to be treated bending of the wall section 160 can occur, for example in order to compensate for unevenness in the skin region or to compensate for skewed placement of the application module 1 (compare bottom illustration in FIG. 15).

FIG. 16 shows illustrations of the application surface 12 for an embodiment of an applicator 9 in which the application element 5 is formed with a needle group 170 within the application element chamber 8. On both sides of the application element chamber 8 the suction chamber 10 extends that comprises several suction openings. In the embodiment shown the application surface 12 is overall of a kidney-shaped design.

FIGS. 17 and 18 show diagrammatic views for the application of the applicator 9 in the region of a zone around the eyes.

FIGS. 19 to 21 each show embodiments of the applicator 9 in top view from below (top illustration) and in cross section (bottom illustration). In particular the cross-sectional views in the bottom part of the respective figure show that the suction chamber 10 comprises a tapered section 190 opposite the suction opening 11. Planar and/or curved wall sections 191, 192 can be provided. In the embodiments shown, the application element 5, which is arranged in the application element chamber 8, is formed with an arrangement of several needles 193.

Finally, FIGS. 22 to 24 show top views of further embodiments of the application surface 12, wherein the application element 5 is in each case formed as a needle group arranged on the lateral edge of the application surface 12.

In FIG. 1 and in FIGS. 19 to 24 darker regions 200 in each case show the position of the tapered section 190. Said tapered section 190 can be arranged so as to be on the outside or on the inside. It can entirely or partly enclose the application element chamber 8 with the viewing direction to the application surface 12.

The features disclosed in the above description, in the claims and in the drawing can be significant both individually and in any combination in the context of realising the various embodiments.

1. An application module for a handheld device for the repeated application of an application element to a human skin or to an animal skin, comprising
a housing,
an application device provided in the housing and comprising an application element arranged on an application element receiving device, wherein the application element receiving device with the application element is displaceable between an extended position and a retracted position,
a coupling device configured to functionally couple to a drive module so that a drive force provided by the drive module can be picked up and provided at least for a drive of the application device,
an applicator, formed on the housing, which applicator in operation comes to rest in a skin region to be treated,
an application element chamber in which in operation the application element is moved forwards and backwards, and which application element chamber comprises an application element opening, associated with the distal end of the application element, which application element opening opens the application element chamber towards the applicator or is arranged in the region of the applicator, and
a suction chamber that is separate from the application element chamber and comprises a suction connection by way of which a pneumatic suction force can be introduced into the suction chamber, wherein a suction opening is provided by way of which the suction chamber opens towards the applicator or which suction opening is arranged in the region of the applicator.

2. The application module according to claim 1, wherein the application element opening and the suction opening are arranged adjacent to each other in the region of an application surface formed on a distal end of the applicator.

3. The application module according to claim 1, wherein the suction chamber is at least in part surrounded by a flared wall section that at least in part on the rear extends away from the suction opening.

4. The application module according to claim 1, wherein the cross section of the suction chamber tapers off in a chamber section opposite the suction opening.

5. The application module according to claim 1, wherein the suction opening is at least in part surrounded by wall sections made from a flexible material.

6. The application module according to claim 1, wherein the application element chamber comprises a lateral opening.

7. The application module according to claim 1, wherein the suction chamber surrounds the application element chamber on several sides.

8. The application module according to claim 1, wherein the coupling device is designed to provide a mechanical, an electrical, a magnetic and/or a pneumatic drive force at least for a drive of the application device.

9. The application module according to claim 1, wherein the coupling device is designed to pick up a rotary and/or a straight-line drive movement.

10. The application module according to claim 1, wherein the coupling device is designed to pick up in a non-contacting manner the drive force provided by the drive module.

11. The application module according to claim 1, wherein the coupling device is connected to a conversion mechanism that is arranged in the housing and that is designed to convert one type of drive movement to some other type of drive movement, which is coupled to the application device, which drive movement differs from the type of drive movement.

12. The application module according to claim 1, wherein in the housing a pumping unit is arranged that is in fluidic communication with the suction connection of the suction chamber in such a manner that a suction pressure generated by the pumping unit can be applied to the suction chamber.

13. The application module according to claim 1, wherein in the housing a fluid reservoir is formed.

14. The application module according to claim 1, wherein the application device is a puncturing device that on the application element receiving device, which is designed as a puncturing-element receiving device, comprises the application element, which is designed as a puncturing element, wherein on the distal end of the puncturing element a puncturing tip is arranged that is configured to puncture the skin.

15. A handheld device for the repeated application of an application element to a human skin or to an animal skin with a drive module, which is designed to provide a drive force, and an application module according to claim 1 that functionally couples to the drive module so that the drive force provided by the drive module can be picked up, and in the application module is provided at least for a drive of an application device.
